(19) United States
(12) Patent Application Publication
(10) Pub. No.: US 2005/0177140 A1
(43) Pub. Date: Aug. 11, 2005

(54) Pulsed light treatment apparatus and associated method with preliminary light pulse generation

(76) Inventor: Harvey Jay, Scarsdale, NY (US)

Correspondence Address: R. Neil Sudol, 714 Colorado Avenue, Bridgeport, CT, 06605-1601, US

(21) Appl. No.: 10/773,834

(22) Filed: Feb. 6, 2004

(51) Int. Cl. A61B 18/20; A61N 5/06

(52) U.S. Cl. 606/9; 607/88

(57) ABSTRACT

A light treatment apparatus includes a light source and a preliminary light generator. The light source is disposed in a casing for generating a predetermined number of primary light pulses of a predetermined first duration, first light intensity, and total energy. The preliminary light generator is mounted to the casing for generating at least one preliminary light pulse having a sufficient intensity to activate a light limiting reaction in protective eyewear of a user prior to the generating of the primary light pulses.

### BACKGROUND OF THE INVENTION

This invention relates to a pulsed light treatment apparatus and also to an associated method.

Pulsed light has been shown to have beneficial effects in the treatment of hair and dermatological conditions. For instance, as discussed in U.S. Pat. No. 6,280,438, hair may be removed from selected skin surfaces by the application of intense, wide area, pulsed electromagnetic energy. U.S. Pat. No. 6,280,438 teaches the use of incoherent polychromatic radiation in a wavelength range that penetrates into the skin without being highly attenuated. U.S. Pat. No. 5,885,273 discloses a method of removing hair that includes producing a plurality of pulses of incoherent electromagnetic energy, which is filtered in accordance with the color of the hair being removed.

The art using electromagnetic radiation such as pulses of incoherent light is intended to permanently remove hair from selected skin surfaces. The light pulses have parameters such as spectral dispersion, pulse duration and total energy that are selected to destroy the hair follicles in the selected skin area. It has been recognized that such methods carry a certain amount of risk that the eyes of the user or operator may be inadvertently damaged. Accordingly, to protect the eyes of the users, as well as the patients and other individuals, companies such as Kentek Corporation and Glendale Protective Technologies are marketing protective goggles or eyeglasses having lenses made of a light-limiting optical material that automatically darkens upon exposure to the light of the pulses. The lenses, for example, of LightSPEED IPL eyewear, are intended to block the damaging light from reaching people's eyes. These lenses also allow the wearer to look through the lens and see normal color and detail when the lenses are in a baseline, non-protective mode. Standard laser or pulsed light protective glasses or goggles are usually colored and thereby do not allow the wearer to accurately visualize the colors, and hence the details, of the treatment sites. Standard laser or pulsed light protective glasses and goggles are therefore frequently removed or elevated by the wearer to enable better and more accurate observation. This removal or elevation is both dangerous (in case of an unexpected pulsed of light) and inconvenient.

A potential problem exists in that the darkening of the lens material experiences a time lag on the order of about 0.3 milliseconds. This brief interval is still long enough to permit some damage to the retinal receptors should a pulse of light be transmitted into the eye.

### OBJECTS OF THE INVENTION

It is an object of the present invention to provide a pulsed-light apparatus and/or an associated method wherein the risk of retinal damage is reduced, if not eliminated.

It is another object of the present invention to provide such a pulsed-light apparatus and/or method adapted to automatically darken protective eyewear.

A further object of the present invention is to provide such a pulsed-light apparatus and/or method that automatically operates to ensure eye safety.

These and other objects of the present invention will be apparent from the drawings and descriptions herein. Although every object of the invention is believed to be achieved by at least one embodiment of the invention, there is not necessarily any one embodiment that achieves all of the objects of the invention.

### SUMMARY OF THE INVENTION

A light treatment apparatus comprises, in accordance with the present invention, a casing, a light source, an applicator element, and a trigger signal generator. The light source is disposed in the casing for generating a predetermined number of light pulses of a predetermined duration, light intensity, and total energy. The applicator element is mounted to the casing in optical communication with the light source for directing light from the source to a target. The signal generator may take the form of a secondary light generator that produces a trigger signal in the form of at least one preliminary light pulse of a predetermined duration and light intensity prior to the generating of the light pulses by the light source. The preliminary light pulse has a sufficient intensity to activate a light limiting reaction in light-limiting optical material prior to the generating of the primary light pulses. The second light intensity is substantially less than the first light intensity and sufficiently low so that the preliminary light pulse poses no substantial risk of damage to retinal receptors.

A control unit is operatively connected to the light source and the preliminary light generator for synchronizing the operation thereof. The control unit times the emission of the preliminary light pulses and the primary light pulses (from the light source) to ensure that a light limiting reaction (darkening) occurs in the optical material in response to the preliminary light pulse to an extent sufficient to effectively block transmission of the primary light pulses through the optical material. The light-limiting reaction of the optical material has a given or known delay from an initial impingement of light on the optical material or on a separate sensor to a point where the material is sufficient darkened to effectively block light transmission. The control unit induces the main light source to initiate the generation of a leading primary light pulse only after a time equal to the delay or reaction time of the light-limiting optical material has passed after the emission of the preliminary light pulse by the preliminary light generator. The primary light pulses may commence at any time while the optical material remains sufficiently darkened to block effective light transmission.

Where the delay or lag time of the light limiting reaction of the optical material is, for instance, three-tenths of a millisecond, the preliminary light pulse begins at least three-tenths of a millisecond prior to the primary light pulses. Where the delay or lag time of the light limiting reaction of the optical material is less, for instance, one-tenth of a millisecond, the preliminary light pulse begins at least one-tenth of a millisecond prior to the primary light pulses.

More preferably, the interval between the firing of the preliminary light pulse and the beginning of the primary light pulses is greater than the delay or lag time in completing the light-limiting reaction of the optical material, i.e., in rendering the optical material opaque to possibly damaging light pulses. Where the refractory period of the optical material is long, for instance, as long as one hundred milliseconds or more, the preliminary light pulse may be commenced one, two, ten or twenty or more milliseconds prior to an onset of the primary light pulses. The preliminary light pulse has an intensity and duration sufficient to activate a sensor or to directly trigger the darkening reaction of the light-limiting optical material, in the case that the darkening reaction is a direct response of the optical material to incident radiation. A preliminary pulse duration equal to the delay or lag time of the light limiting reaction of the optical material is generally effective. However, shorter or longer durations may also be effective. For instance, where a dedicated sensor is provided for detecting the preliminary light pulse(s), the duration of the preliminary pulse(s) need be only long enough to energize the sensor.

Where the light source is a primary light source, the preliminary light generator may include a secondary light source such as one or more light emitting diodes different from the primary light source. The secondary light source or sources may be disposed at locations remote from the primary light source and the control unit. The communications links between the control unit and the secondary light sources may be hard wired or alternatively wireless.

In one embodiment of the present invention, the trigger signal may itself be a wireless RF, infrared, or microwave signal or an ultrasonic pressure wave. In that case, the light-limiting reaction in the optical material is induced by subjecting the optical material to a predetermined voltage or electrical current in response to the reception of the trigger signal by a sensor.

The primary light pulses may be greater than one in number and have at least one predetermined inter-pulse interval longer than the refractory period of the light-limiting optical material. In that case, the trigger signal (e.g., preliminary light pulse) may be one of a plurality of trigger signals (preliminary light pulses) produced by the signal generator, each of the trigger signals beginning prior to a respective one of the primary light pulses by a time at least equal to the delay or lag time of the optical limiting reaction.

Where the applicator element is adapted to direct light in a first direction towards the target area, a preliminary light generator is preferably adapted to direct light in at least one second direction different from the first direction. Pursuant to this feature of the invention, a preliminary light pulse is preferably a substantially omni-directional emission, intended to activate protective light-absorbing lenses regardless of the location of the wearer relative to the direction of pulsed light application. Thus, the eyes are protected in the case of unanticipated reflections or refractions, as well as direct transmissions along the direction of pulse-light application.

An associated light treatment method in accordance with the present invention comprises (a) generating a predetermined number of primary light pulses of a predetermined duration, light intensity, and total energy, (b) directing the light pulses to a target, and (c) generating at least one trigger signal for inducing the generation of a ligh-limiting reaction in optical material.

As discussed above, the trigger signal may be a preliminary light pulse of a predetermined duration and light intensity. The preliminary light pulse has a sufficient intensity to directly or indirectly activate a light limiting reaction in optical material, for instance, in protective eyewear of a user, prior to the generating of the primary light pulses. However, the intensity of the preliminary light pulse is substantially less than the intensity of the primary light pulses and sufficiently low so that the preliminary light pulse poses no substantial risk of damage to retinal receptors.

The generating of the primary light pulses generally includes operating a first light source, while the generating of the preliminary light pulse includes operating a second light source different from the first light source. A control unit operatively connected to the light sources undertakes the operating of the light sources. However, it is possible, for instance, to produce the preliminary light pulse from light generated by the primary light source. A filter may be used to diminish the intensity of the light for a predetermined period of time prior to the onset of the primary light pulses. That period of time must be at least equal to the delay or lag time of the light-limiting reaction of the optical material.

### BRIEF DESCRIPTION OF THE DRAWINGS

FIG. 1 is a block diagram of a light-pulse generating apparatus in accordance with the present invention, for use in a method in accordance with the present invention.

FIG. 2 is a block diagram of another light-pulse generating apparatus in accordance with the present invention, for use in a method in accordance with the present invention.

FIG. 3 is partially a schematic side elevational view of goggles and partially a block diagram showing a modification of the apparatus of FIG. 2.

### DEFINITIONS

The term “light-limiting optical material” as used herein denotes a material that is transparent at ambient light levels but may be darkened to an essentially opaque state. The darkening reaction may be induced by the application of an electrical potential across the light-limiting optical material. Alternatively, the darkening reaction may be induced by the falling of pulsed light energy on the optical material itself. In the former case, the light-limiting reaction may be triggered by a photocell or other sensor such as a wireless receiver that receives an appropriate activation signal. In the latter case, a light-limiting reaction is triggered directly in the optical material by a sudden increase in the intensity of incoming electromagnetic radiation. In either case, a preliminary light pulse in the form of a light flash from a diode or other source of incoherent radiant (electromagnetic wave) energy may trigger the light-limiting reaction. Alternatively, but not preferably, a laser pulse or other kind of wireless (radio wave, ultrasonic) signal may trigger the light limiting reaction. As described herein, a preliminary light pulse used to trigger a darkening reaction in light-limiting material, for instance, of protective goggles or eyeglasses, is sufficiently intense and of sufficient duration to trigger or induce the light-limiting reaction but is not so intense as to damage retinal tissues. A light-limiting optical material as that term is used herein may be a polymer or plastic, a gel or a cream, or other solid or fluidic composition.

The term “delay” or “lag” is used herein with reference to a light-limiting optical material to denote the time from a commencement of a triggering signal, such as a light pulse incident on the material, to a darkening of the material effective to prevent transmission of radiation that is damaging to retinal and/or other organic tissues. Current commercial versions of light-limiting optical material have a delay time of the light-limiting reaction on the order of three-tenths of a millisecond from the time that an initial burst of light impinges on a light sensor to the time that the light limiting reactino in the optical material is sufficient to prevent the transmission of radiation through the optical material.

The term “preliminary light pulse” is used herein to denote a pulse of radiant energy used solely for the purpose of triggering a darkening reaction in light-limiting optical material. A preliminary light pulse is of sufficient intensity and duration to activate a photocell or other optical sensor or to directly stimulate the light-limiting function of the optical material but does not convey enough energy to damage retinal or other organic tissues. A preliminary light pulse may have a duration less than, equal to or even greater than the delay or lag time of the light-limiting reaction of the optical material. In any case, a preliminary light pulse precedes a respective primary light pulse by a time greater than the delay of lag time of the light-limiting optical material.

The term “refractory period” as used herein with reference to a light-limiting optical material denotes the time required for the resumption of normal light transmissivity after a blocking or darkening reaction. More specifically, the term “refractory period” is used herein to denote the time interval extending from the cessation of pulsed light incident on the optical material to a state that the optical material is capable of transmitting an amount or intensity of radiation that is potentially dangerous to retinal or other organic tissues.

The term “primary light pulse” refers generally herein to a pulse of light energy used to achieve a desired result other than triggering a darkening reaction in light-limiting optical material. A primary light pulse may be used, for instance, to effectuate a therapeutic result in skin tissues, hair, or vascular tissues. A primary light pulse may be used experimentally in the laboratory to examine the effects of radiant energy on various biological, microbiological, histological, cytological, chemical, or semiconductor, materials, etc.

The term “applicator element” as used herein denotes a light guide for channeling radiant energy in a desired direction from a light source to a target. A light applicator may be an optical element such as a mirror, lens, or prism, or a light transmitting member such as a fluid-filled sac or bag, a block of hydrogel material, an optical fiber or bundles of optical fibers, etc.

The term “blocking” is used herein to denote a state of a light-limiting optical material wherein the material is darkened sufficiently to prevent the transmission of amounts or intensities of electromagnetic radiation that would be damaging to retinal and/or other organic tissues.

### DETAILED DESCRIPTION OF THE PREFERRED EMBODIMENTS

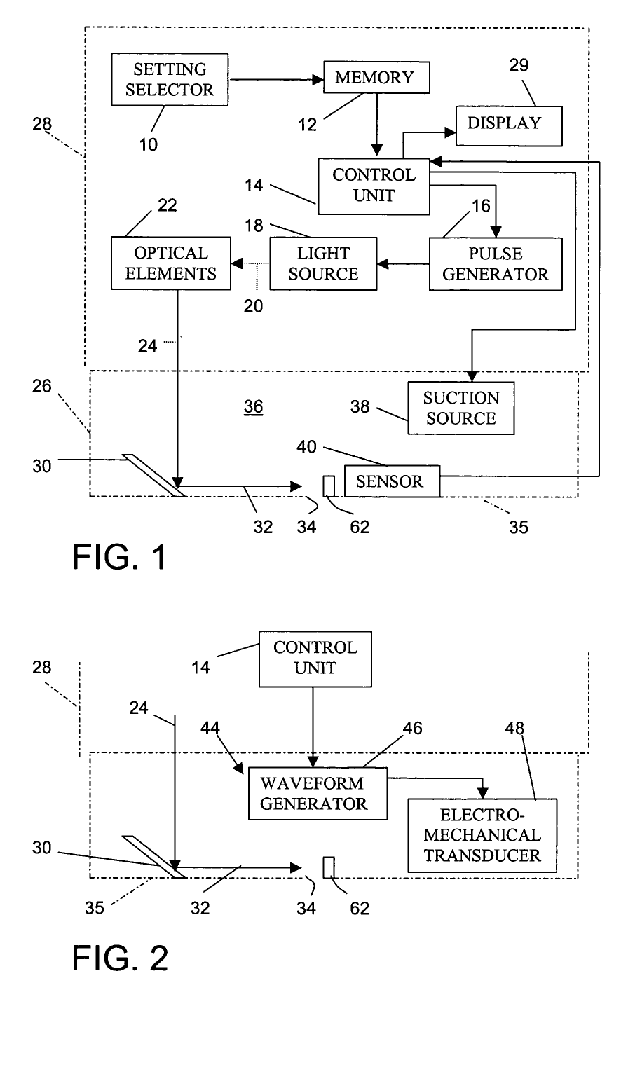

As depicted in FIG. 1, a device for generating light pulses for application to a skin surface in a hair or skin treatment process includes a manually operable setting selector 10 connected at an output to a memory 12 in turn connected at an output to a control unit 14. Memory 12 stores pre-established combinations of primary light pulse parameters including pulse width or duration, inter-pulse interval or delay time, pulse number, light intensity, and total treatment energy. Control unit 14 may be a microprocessor or a special logic circuit connected to a pulse generator 16 for inducing the generator to produce a sequence of electrical control pulses fed to a source 18 of incoherent light energy. Source 18 produces light with a spectral distribution including wavelengths between 500 nm and 1200 nm. Control unit 14 may be connected directly to source 18 where the source incorporates means for varying pulse parameters pursuant to encoded instructions.

Light source 18 (as well as the entire light pulse applicator) may take any known form such as those disclosed in U.S. Pat. No. 6,280,438 and U.S. Pat. No. 5,885,273. Thus, light source 18 may be a Xenon flashlamp.

Light 20 generated by source 18 is directed through an array of optical elements 22 that may include one or more reflectors, lenses, and filters (not separately shown). Where an adjustable filter is included, control unit 14 may be connected to the filter for operatively modifying the action thereof. For instance, in the case of an adjustable neutral density filter, control unit 14 may induce a change in the filter density to control the intensity, and therefore the power, of the light applied to a selected skin surface.

In the case of multiple wavelengths of light being produced, an adjustable filter may be included in the optical elements 22 and/or the applicator interface 26. These filters can block undesired wavelengths and allow desired wavelengths to pass. Low end filters that block lower or shorter wavelengths, high end filters that block higher or longer wavelengths or band pass filters that block some high or some low end wavelengths may be utilized.

Light 24 leaving the optical array 22 is delivered or applied to a skin surface via an applicator or interface element 26 exemplarily taking the form of a crystal, a hydrogel block, or a pouch filled with a fluid or a gel. U.S. Pat. No. 6,280,438 and U.S. Pat. No. 5,885,273 disclose kinds of applicators or interfaces utilizable in the device of FIG. 1 (or 2). Applicator or interface element 26 may function in part to cool the skin surface prior to, during, and/or after a light application procedure. Cooling may be accomplished by using a crystal-type applicator or interface 26, with or without a layer of gel, as described in U.S. Pat. No. 6,280,438 and U.S. Pat. No. 5,885,273. Alternatively or additionally, cooling may be accomplished by spraying a coolant on the skin surface or by blowing air or other gas on the skin surface. In the former case, the light application device is provided with a reservoir of coolant fluid, an ejection mechanism or pump and a nozzle. In the latter case, the device is provided with a pump or compressor and a nozzle for directing a jet of air at the skin surface being treated. The elements of FIG. 1 are encased in or mounted to a housing or casing 28 of a size and configuration enabling the pulse generation device to be hand held and easily manipulated for purposes of optically treating different skin surfaces of the individual user.

The device of FIG. 1 is preprogrammed to produce light pulses in any of several settings, each setting being defined by a respective combination of particular operational parameters including pulse duration, inter-pulse interval, pulse number, and intensity or total energy. For instance, the device may have a plurality of settings, for instance, high, medium, and low, which vary in the number of applied pulses (e.g., 3, 2, 1), the pulse duration (9 msec, 7 msec, 5 msec), the inter-pulse interval (250 msec, 300 msec, 350 msec), and/or the total energy applied (35 J/cm², 20 J/cm², 10 J/cm²). A user could start with a low setting to see whether a desired result is achieved and, if not, try the next higher setting. Usually, it is preferable to use the lowest setting which accomplishes the desired result. The desired result may be temporary or permanent hair removal, hair growth stimulation, skin rejuvenation, cancer inhibition, etc.

The light treatment apparatus of FIG. 1 further comprises a preliminary light generator 25 including a pulse generator 27 operatively connected at an input to control unit 14 and at an output to a light source 29 in the form of at least one light emitting diode (LED). Preliminary light generator 25 is mounted to casing or housing 28 for generating, in response to signals from control unit 14, at least one preliminary light pulse of a predetermined duration, light intensity, and total energy prior to the generating of the primary light pulses by light source 18. The preliminary light pulse has a sufficient intensity to directly or indirectly activate a light limiting reaction in light-limiting optical material of protective eyewear prior to the generating of the primary light pulses by light source 18. In the case of indirect activation, the preliminary light pulse is detected by a photosensor located, for instance, on the frame of the protective eyewear. The reception of the preliminary light pulse by the photosensor results in the application of a voltage or the conduction of electrical current across the lens material of the protective eyewear, causing the lens material to darken. In the case of direct activation of the light-limiting reaction of the optical material, the protective eyewear is of a known type having lens material that experiences a reduction in optical transmissivity upon the absorption of incoherent light energy.

The light intensity of the preliminary light pulse emitted by LED 29 is substantially less than the intensity of the primary light pulses emitted via applicator 26 and sufficiently low so that the preliminary light pulse poses no substantial risk of damage to retinal receptors.

LED 29 may be one of a plurality of similarly functioning diodes and emits an essentially onmi-directional electromagnetic waveform to induce the light limiting reaction in protective eyewear worn by a user, an operator, an assistant, an observer, or a patient in the vicinity of the device of FIG. 1. Thus, where applicator element 26 is adapted to direct light in a first direction towards a target skin area, LED 29 is preferably adapted to direct light in at least one direction different from the first direction.

Control unit 14 synchronizes the operation of light sources 18 and 29 to ensure that a light limiting reaction (darkening) occurs in the optical lens material as a result of the preliminary light pulse to an extent sufficient to effectively block transmission of the primary light pulses through the optical material. Thus, the eyes behind the protective goggles or eyewear with lenses made of the light-limiting optical material are protected.

Control unit 14 induces the main light source 18 to initiate the generation of a leading primary light pulse only after the preliminary light generator 25 has emitted the preliminary pulse. The preliminary pulse should precede the primary pulse by a time equal to or greater than the lag time of the light-limiting optical material. The primary light pulses may commence at any time within the blocking or refractory period of the optical material, i.e., within the time that the optical material remains sufficiently darkened to block effective light transmission. In current materials, this refractory period may be as long as 0.5 second.

Where the delay or lag time of the light limiting reaction of the optical material is, for instance, three-tenths of a millisecond, control unit 14 induces preliminary light generator 25 to initiate generation of the preliminary light pulse at least three-tenths of a millisecond prior to an onset (increasing light intensity) of the primary light pulses. Control unit 14 may commence preliminary light pulse generation at a time even more advanced with respect to the onset of the primary light pulses. Where the refractory period of the optical material is long, for instance, as long as one hundred milliseconds or more, the preliminary light pulse may be commenced one, two, ten or twenty or more milliseconds prior to an onset of the primary light pulses. The preliminary light pulse may have a duration less than or equal to the delay or lag time of the light limiting reaction of the optical material and may terminate as late as the end of the primary pulse sequence.

Where the delay or lag time of the light-limiting reaction of the optical lens material is three-tenths of a millisecond, the preliminary light pulse emitted by LED 29 of light generator 25 begins at least three-tenths of a millisecond prior to an onset (increasing light intensity) of the primary light pulses produced by light source 18 and emitted via applicator element 26. This minimum time interval could conceivably be shorter particularly in the event that the response time of the darkening lens material for pulsed light applications is reduced to less than three-tenths of a millisecond. It is preferable, however, to provide a safety factor and have the preliminary light pulse commenced earlier. A satisfactory interval would be if the preliminary light pulse were to begin at least one millisecond prior to an onset of the primary light pulses. A preliminary light pulse having even an earlier onset, perhaps several milliseconds or tens of milliseconds in advance of the primary pulses, would be even safer. In no event, however, may a preliminary light pulse terminate before a respective primary light pulse by a time interval greater than the refractory period of the light-limiting optical material.

Where a plurality of primary light pulses are generated by source 18 and the inter-pulse interval is greater than the refractory period of the light-limiting optical material of protective eyewear, control unit 14 may induce preliminary light generator 25 to emit a like plurality of preliminary light pulses each having an onset prior to the onset of a respective one of the primary light pulses. Thus, each skin treatment pulse is immediately preceded, e.g., by three-tenths of a millisecond, by its own preliminary light pulse.

The function of preliminary light generator 25 may be performed alternatively by primary light source 18 and optical elements 22. More particularly, a neutral density filter (not specifically shown) included in the optical elements may be activated to produce a preliminary light pulse. Control unit 14 operates the filter for a predetermined period, preceding the primary pulse by at least three-tenths of a millisecond, at the beginning of each primary pulse to reduce the emitted light intensity to a safe level. Should there be a light transmission path from applicator element 26 to a person's eye, the reduced intensity light at the onset of each pulse would trigger the light limiting effect of protective eyewear sufficiently in advance of the full intensity light emission to ensure adequate eye protection. In this case, the neutral density filter of optical elements 22 functions as a preliminary light pulse generator while the reduced intensity portion of the light pulses can be understood to be a separate light pulse.

Alternatively or additionally to the neutral density filter, optical elements 22 may include a light scattering element (not shown) for temporarily directing the light from source 18 in an effectively omnidirectional pattern. The filter may be unnecessary in this case since the intensity of the emitted light over a given unit of flux area is reduced owing to the spreading of the light. To generate the preliminary light pulse, optical elements 22 may include a reflector or other component for shifting a light transmission path to include the filter and/or the scattering element.

A more advanced or complex device for effectuating such objects as temporary or permanent hair removal, hair growth stimulation, skin rejuvenation, cancer inhibition, etc., is illustrated in FIG. 2. This device includes a housing or casing 30 having manually actuatable input elements 32, 34, 36, and 38, such as rotary knobs or a solid-state touch screen, which enable a user to individually select multiple operating parameters. Input elements or selectors 32, 34, 36, and 38 are an inter-pulse interval selector, a pulse number selector, a power or energy selector, and a pulse duration selection, respectively. Another selector (not shown) could be for intensity adjustment, while a further selector may be provided for adjusting a light source 42 or a filter in optical elements 48 and/or an applicator 52 for modifying the wavelength band delivered to the target skin surface. Selectors 32, 34, 36, and 38 are operatively tied to a control unit 40 such as a microprocessor or hard-wired log circuit. Control unit 40 regulates the operation of light source 42 such as a conventional flashlamp, either directly or indirectly via a pulse generator 44. Light 46 from source 42 is transmitted along a path through optical elements 48 optionally including one or more reflectors, lenses, and filters (not separately shown). Light 50 at an output of the optical array 48 is applied to a skin surface via applicator or interface element 52. Applicator or interface element 52 may take the form of a crystal block, a flexible polymeric (e.g., hydrogel) element, and/or a transparent or translucent pouch filled with a transparent or translucent fluid such as a gel or a liquid. In the case of the flexible applicator element or the fluid-filled pouch, applicator or interface element 52 conforms at least partially to the changing topography of the skin surface under treatment and may thereby facilitate the retention of gel, if any, between the applicator or interface 52 and the skin surface. This result decreases the likelihood of overexposed or burned skin and generally provides a more uniform application of light with a uniformity of cooling. Safety is enhanced, while the outcomes to successive procedures become increasingly standardized.

As an alternative to the flexible applicator or fluid-filled pouch, applicator or interface element 52 may include a plurality of independently movable substantially rigid transparent or translucent members (not shown) that collectively define a tissue-engaging surface. These independently movable members may take the form of closely packed pins or plates that are each independently spring biased to an extended position. Pressure of topographical dermal features against the independently movable pins or plates during use of the light-pulse generating device causes the pins or plates to move in opposition to the respective spring bias, to thereby conform the tissue engaging surface of the light-pulse generating device to the skin surface under treatment. The independently movable pins or plates may be disposed in a holder or bracket attached to the housing or casing 30 and retained there by friction forces.

Where applicator 52 (or 26) includes a gel-filled pouch, the pouch (52) may be provided with perforations on a skin-contacting surface for exuding the gel for cooling purposes. Alternatively, as shown in FIG. 2, the light pulse device may be provided with a fluid dispenser such as a spray nozzle 54 connected to a valve 56 downstream of a pressurized coolant reservoir 58. In response to an operation of a manual actuator 60 or in response to signals from control unit 40, valve 56 enables a flow of coolant from reservoir 58 to nozzle 54 for application to a selected skin surface. In the event that applicator or interface element 52 is a bag or pouch, reservoir 58 and valve 56 may be connected to the applicator or interface element for supplying a gel or fluid coolant thereto.

The light treatment apparatus of FIG. 2 is provided with a preliminary light generator 64 including a pulse generator 66 and a light source 68 in the form of at least one light emitting diode (LED). Pulse generator 66 is operatively connected at an input to control unit 40 and at an output to LED 68. Preliminary light generator 64 is attached to casing or housing 30 and serves to produce, under the control of unit 40, at least one preliminary light pulse of a predetermined duration, light intensity, and total energy. LED 68 emits the preliminary light pulse prior to the transmission of the primary light pulses from light source 42 to a target skin surface via applicator element 52. The preliminary light pulse has a sufficient intensity to directly or indirectly activate a light limiting reaction in protective eyewear. The eyewear has lens material that may be temporarily rendered effectively opaque, either by the application of an electrical voltage or current or by the reception of light energy having a sufficient intensity. The light intensity of the preliminary light pulse emitted by LED 68 is substantially less than the intensity of the primary light pulses emitted via applicator 52 and sufficiently low so that the preliminary light pulse poses no substantial risk of damage to retinal receptors.

LED 68 includes one or more similarly functioning diode elements that emit electromagnetic radiation in an essentially omni-directional pattern to induce the light limiting reaction in protective eyewear in a space about the device of FIG. 2. Thus, where applicator element 52 is adapted to direct light in a first direction towards a target skin area, LED 68 is preferably adapted to direct light in at least one direction different from the first direction.

The preliminary light pulse emitted by LED 68 of light generator 64 begins prior to an onset (increasing light intensity) of the primary light pulses produced by light source 42 and emitted via applicator element 52. Control unit 40 synchronizes the operation of light source 42 and preliminary light pulse generator 64 to ensure that a light limiting reaction (darkening) occurs in the optical lens material directly or indirectly in response to the preliminary light pulse to an extent sufficient to effectively block transmission of the primary light pulses through the optical material. Thus, the eyes behind the protective goggles or eyewear with lenses made of the light-limiting optical material are protected.

Control unit 40 induces the main light source 42 to initiate the generation of a leading primary light pulse only after the preliminary light generator 64 has emitted the preliminary pulse. The preliminary pulse should precede the primary pulse by a time equal to or greater than the lag time of the light-limiting optical material. The primary light pulses may commence at any time within the blocking or refractory period of the optical material, i.e., within the time that the optical material remains sufficiently darkened to block effective light transmission.

Where the delay or lag time of the light limiting reaction of the optical material is, for instance, three-tenths of a millisecond, control unit 40 induces preliminary light generator 64 to initiate generation of the preliminary light pulse at least three-tenths of a millisecond prior to an onset (increasing light intensity) of the primary light pulses. Control unit 40 may commence preliminary light pulse generation at a time even more advanced with respect to the onset of the primary light pulses. Where the refractory period of the optical material is long, for instance, as long as one hundred milliseconds or more, the preliminary light pulse may be commenced one, two, ten or twenty or more milliseconds prior to an onset of the primary light pulses. The preliminary light pulse may have a duration equal to the delay or lag time of the light limiting reaction of the optical material.

Control unit 40 may induce preliminary light generator 64 to emit a separate preliminary light pulse for each pulse in a primary sequence of skin treatment pulses. This is particularly useful in the event that the inter-pulse interval of the treatment pulse sequence is longer than the refractory period of the light-limiting lens material of protective eyewear worn by people in the vicinity of the light treatment device. In that case, each preliminary light pulse begins an effective interval, for instance, three tenths of a millisecond or more, before the respective skin treatment pulse.

Alternatively, a neutral density filter (not specifically shown) included in optical elements 48 may perform the function of preliminary light generator 64. Under the control of unit 40, the filter reduces the light intensity at the beginning of each primary pulse to reduce the emitted light intensity to a safe level for a predetermined period (at least three-tenths of a millisecond for the current state of the art). This reduced intensity pulse at the onset of each treatment pulse will trigger the light limiting effect of any protective eyewear in the vicinity sufficiently in advance of the full intensity light emission to ensure adequate eye protection. Alternatively or additionally to the neutral density filter, optical elements 48 may include a light scattering element (not shown) for temporarily directing the light from source 42 in an effectively omnidirectional pattern. To generate the preliminary light pulse, optical elements 48 may include a reflector or other component for shifting a light transmission path to include the filter and/or the scattering element.

In one embodiment of the device of FIG. 2, suitable for professional but not home use, inter-pulse interval selector 32 provides for intervals in a range from 1 msec and 2 seconds, whereas pulse number selector 34 is enabled for pulse sequences of one to ten pulses. In addition, power selector 36 permits treatment energies between 1 Joule per square centimeter of skin surface and 200 Joules per square centimeter, while pulse duration selector 38 enables pulses of 1 msec to 2 seconds in length. Total pulse sequence duration, from the beginning of the first pulse to the termination of the final pulse, ranges from 1 msec to 38 seconds. The various parameters of a primary or therapeutic pulse sequence may be selectable from sets of discrete values or, alternatively, from continuous ranges.

In the device of FIG. 2, the various parameters of a primary or treatment pulse sequence are typically not completely independent inasmuch as the total energy selected will function as a constraint on the ranges available for the other parameters, that is, the total energy selected will serve to regulate or circumscribe the ranges available to the user for the other pulse sequence parameters. Where the device of FIG. 2 has no intensity adjustment capability, a selection of the total energy and the pulse duration may determine the number of pulses. Similarly, a selection of the total energy and the number of pulses may determine the pulse duration. If the intensity is an adjustable parameter, once the total energy has been chosen, the user will be able to select the magnitudes of two of the three parameters, pulse duration, intensity and number of pulses. The inter-pulse interval is related to the rate at which radiant energy is applied to a skin surface and may accordingly be subjected to some programmed control. Longer pulse durations and/or delays will deliver energy at a slower rate (total energy is distributed over longer time) and therefore be safer to use with higher energy levels. Preferably, the total energy is always a selectable parameter and is best selected prior to the setting of the other parameters. However, the device of FIG. 2 may be preprogrammed to limit the rate at which radiant energy is applied to a skin surface, which will force restrictions on the user's ability to select pulse parameter values.

In an alternative embodiment of the device of FIG. 2, suitable for home use, inter-pulse interval selector 32 enables a selection of intervals ranging from 200 msec to 2 seconds, while power selector 36 enables treatment energies between 1 J/cm² and 40 J/cm². Preferably, the pulse duration and the number of pulses available for selection are restricted so as to prevent the user from delivering energy at too high a rate. If the user selects a large pulse number, the pulse duration is necessarily short, whereas a small number of pulses forces a longer pulse duration in order to achieve the selected total energy. It is preferable to use such a number of pulses and such a pulse duration as to limit the rate at which light energy is applied to a skin surface. Pulse number selector 34 may therefore enable a selection of three to ten pulses per pulse sequence, while pulse duration selector 38 enables a selection of pulses lasting 1 msec to 10 msec. The various pulse sequence parameters may be selectable from sets of discrete values or, alternatively, from continuous ranges.

A person uses the device of FIG. 1 or 2 to apply pulses of light to a skin surface, for instance, for purposes of effectively severing or destroying hair fibers below the surface of the skin to temporarily prevent hairs from growing through and thus becoming visible on the skin. The user first performs a calibration or initialization procedure to determine an appropriate pulse setting and a hair-regeneration period for that setting. The term “hair-regeneration period” is used herein to denote the time it takes for hair to reappear on the skin surface after a pulse sequence has been applied to that surface at a selected setting.

During a calibration or initialization stage of a temporary hair removal method, the user should first select a low-energy pulse sequence to determine whether that sequence is effective in removing the hair of a selected skin region. The individual may find that a given setting does not adequately remove the hair (e.g., some hairs do not fall out) or requires a too frequent treatment. In such cases, the individual should retry the calibration or initialization procedure using a higher-energy setting.

Using the device of FIG. 1, for instance, for temporary hair removal, an individual will first select a low setting to determine whether that low setting is effective in hair removal. If not, a next higher or medium setting may be tried. Generally, higher settings will be used only as the circumstances warrant, for instance, if the hair fibers are thick and the skin is light.

In determining optimal settings with the device of FIG. 2, a user should choose initial parameter values which in combination result in the application of small amounts of energy. Thus, where one or more selected pulse parameters are associated with high treatment energies, other pulse parameters should be selected that are associated with low treatment energies.

Where the total applied energy is allowed to decrease (e.g., to less than 40 Joules per square centimeter of skin surface) while other pulse parameters are held constant, lower average rates of energy application result from reducing the number of pulses (e.g., from 8-10 to 1-3 pulses), increasing the inter-pulse intervals (e.g., to 300 msec or more), decreasing the pulse durations (e.g., to 20 msec or less), and reducing the light intensity (if selectable, for example, via an adjustable neutral density filter). If a given setting proves to be ineffective, the user might adjust selector 32 or 38 to decrease the inter-pulse interval or increase the pulse length, thereby effectively increasing the power or rate at which the radiant energy is delivered to the target skin surface. Alternatively or additionally, the user might increase the number of pulses via selector 34 or increase the applied energy via selector 36. These adjustments will result in an increase in the rate of applied energy if the total time of the pulse sequence is limited. If the light intensity is separately adjustable, one may increase the power or rate of energy delivery by simply selecting a higher intensity value.

Where the various pulse parameters are not independently selectable, for instance, where the total energy applied is a controlling factor, adjustments made in the parameters for purposes of incrementally enhancing the effectiveness of the device of FIG. 2 will be different from the case of completely independent parameter values. For instance, once the total applied energy and total pulse sequence time have been selected, decreasing the number of pulses will require an increase in pulse length and/or an increase in pulse intensity in order to deliver the same amount of total energy in the fixed time. These changes will increase the effectiveness of the light application inasmuch as the rate of energy delivery is increased. In contrast, once the total applied energy and total pulse sequence time have been selected, increasing the pulse duration will decrease the instantaneous rate at which energy is applied to the target skin surface by decreasing the light intensity.

During the calibration or initialization stage of a hair removal method using the device of FIG. 1 or FIG. 2, light is used on skin surfaces with visible and protruding hair. Light is applied to the skin surface and the hair and is directed downward towards the base or bulb of the hair. Immediate damage to the hair may be noted but is not essential. Hairs may fall out during the course of the following month. Hair loss may be gradual or abrupt. No assistance is usually needed in this process.

With regard to the use of the devices of FIGS. 1 and 2 for hair removal, it is to be noted that hair growth rates vary from person to person and for different body locations on the same person. Consequently each user should note the interval between the first treatment and the reappearance of new hair on each skin area. In addition, because different skin areas have different grades of hair (different colors, different fiber diameters, different hair densities) and different skin pigmentation, etc., different pulse parameter settings are recommended for different skin areas. For example, different settings will be necessary for the underarms and the legs in order to optimize results. In addition, depilation schedules may also vary from one skin area to another.

After the user has determined appropriate settings of the pulse sequence parameters and expected hair-regeneration periods for different skin areas, the user then treats each skin surface with pulsed light at the respective setting and at a periodicity set by the respective hair-regeneration period. Successive applications of pulsed light follow at intervals smaller than the detected hair-regeneration period. For instance, if it is determined that hair reappears on a leg at three weeks after treatment with light at a given pulse sequence setting, then light energy at that setting is applied to the leg at, say, two week intervals to maintain the leg free of visible hair. The regeneration period may be measured again after any number of treatments. And if the user finds that the regeneration time has changed, the interval between successive treatment sessions may be adjusted accordingly.

The following discussion applies particularly to the use of the devices of FIGS. 1 and 2 in a temporary hair removal method. The method contemplates the periodic application to a selected skin surface of a pulse sequence having a predetermined number of pulses of light of a predetermined electromagnetic spectrum, a predetermined duration, a predetermined inter-pulse interval, and a predetermined total energy. These pulse sequence parameters are determined in part by the design of the light-generating device used and in part by the selections made by the user. The light treatment temporarily prevents a growth of hair through the selected skin surface for the respective hair-regeneration period.

The light of the pulses is generally incoherent and the spectrum includes wavelengths between about 300 nm and 1200 nm. However, single wavelengths of laser or coherent light may be delivered at one time, when desired. Higher wavelengths are used for darker skin, for deeper hairs and deeper removal. In order to limit the depth of penetration of the light, and accordingly the length of the hair-regeneration or hair-regrowth period, the spectrum of the pulses may be limited to shorter wavelengths and may include wavelengths, for instance, below 550 nm.

The light applied to a skin surface by the devices of FIGS. 1 and 2 may include at least one wavelength absorbable by an endogenous chromophore in a person's hair. The endogenous chromophore may be a form of melanin such as pheomelanin or eumelanin. In a more advanced embodiment the light application device may include a setting or control (not shown) for selecting a spectrum or range of wavelengths appropriate to the user's hair color. For instance, for lighter hair, the wavelengths selected encompass one or more natural absorption wavelengths of pheomelanin. For darker hair, the wavelengths selected encompass one or more natural absorption wavelengths of eumelanin. In any event, the devices of FIGS. 1 and 2 are used without the application of an exogenous chromophore to a target skin surface for light absorption purposes. Hair removal and growth retardation are accomplished by light absorption solely by one or more endogenous chromophores.

In other embodiments of a light generation and application device for hair treatment, one or more of the pulse parameters may vary during a single treatment session. For instance, the inter-pulse interval or the pulse duration may increase or decrease from the beginning of a pulse sequence to the end of the pulse sequence. The resulting instantaneous rate of energy application may therefore vary during the pulse sequence.

Listed below are a number of exemplary settings or combinations of operational parameters particularly suitable for home-use and attainable with either the device of FIG. 1 having pre-established settings or parameter combinations or the device of FIG. 2 where the various pulse sequence parameters may be individually adjusted independently of the other parameters. In these examples, the total times of the pulse sequences are determined by the selected numbers of pulses, the selected pulse durations and the selected inter-pulse intervals. The light intensity may be automatically adjusted by the light generating device if necessary to ensure consistency among the listed parameter settings.

### HOME USE EXAMPLE 1

In a preferred setting or combination of operational parameters suitable for home use, an incoherent light applicator device for temporary hair removal generates pulses with a pulse number of two, a pulse duration of 7 msec, an inter-pulse interval of 300 msec, a total pulse energy of 20 J/cm², and a spectral distribution of a commercially available flashlamp, including wavelengths between 500 and 1200 nm.

### HOME USE EXAMPLE 2

A slightly higher setting or combination of operational parameters for an incoherent light applicator device suitable for home use involves a pulse sequence with a pulse number of two, a pulse duration of 7 msec, an inter-pulse interval of 250 msec, a total pulse energy of 20 J/cm², and a spectral distribution of a commercially available flashlamp, including wavelengths between 500 and 1200 nm. Although the total amount of energy is the same as in the first example, the shorter interpulse interval means that the rate of energy transmission to the target skin surface is higher.

### HOME USE EXAMPLE 3

A higher setting or combination of operational parameters for an incoherent light applicator device involves pulses with a pulse number of two, a pulse duration of 5 msec, an inter-pulse interval of 250 msec, a total pulse energy of 25 J/cm², and a spectral distribution of a commercially available flashlamp, including wavelengths between 500 and 1200 nm. In this example, not only is the total energy larger than in the second example, but the rate of energy application is higher owing to the shorter pulse duration.

### HOME USE EXAMPLE 4

An even higher setting or combination of operational parameters for an incoherent light applicator device involves pulses with a pulse number of two, a pulse duration of 5 msec, an inter-pulse interval of 210 msec, a total pulse energy of 37 J/cm², and a spectral distribution of a commercially available flashlamp, including wavelengths between 500 and 1200 nm. The pulse sequence of this example delivers radiant energy at a higher rate than in the third example because of the shorter inter-pulse interval and the slightly higher energy delivered per pulse.

### HOME USE EXAMPLE 5

In a low setting or combination of operational parameters, an incoherent light applicator device produces pulses with a pulse number of two, a pulse duration of 5 msec, an inter-pulse interval of 350 msec, a total pulse energy of 15 J/cm², and a spectral distribution of a commercially available flashlamp, including wavelengths between 500 and 1200 nm. The pulse sequence of this example delivers a small amount of energy, at a low rate (e.g., long inter-pulse interval).

### HOME USE EXAMPLE 6

A slightly higher setting or combination of operational parameters for an incoherent light applicator device involves pulses with a pulse number of two, a pulse duration of 5 msec, an inter-pulse interval of 300 msec, a total pulse energy of 20 J/cm², and a spectral distribution of a commercially available flashlamp, including wavelengths between 500 and 1200 nm.

### HOME USE EXAMPLE 7

A lower setting or combination of operational parameters for an incoherent light applicator device involves pulses with a pulse number of three, a pulse duration of 5 msec, an inter-pulse interval of 300 msec, a total pulse energy of 20 J/cm², and a spectral distribution of a commercially available flashlamp, including wavelengths between 500 and 1200 nm.

### HOME USE EXAMPLE 8

Another setting or combination of operational parameters for an incoherent light applicator device involves pulses with a pulse number of two, a pulse duration of 10 msec, an inter-pulse interval of 400 msec, a total pulse energy of 20 J/cm², and a spectral distribution of a commercially available flashlamp, including wavelengths between 500 and 1200 nm.

The devices of FIGS. 1 and 2 may be provided with a low-pass filter, a band-pass filter, or a high-pass filter. A band-pass filter operates to limit the spectral distribution of the generated light pulses to wavelengths in a given band, for instance, between 700 nm and 900 nm. A low-pass filter may be used for transmitting to a skin surface only wavelengths less than a predetermined maximum, such as 900 nm, 750 nm, or 550 nm. The lower the wavelength the less likely the light will penetrate deeply and damage cellular and histological elements as deep as the bulb parts of the hair follicles. Shorter wavelengths, for instance, below 550 nm are useful for limiting the depth of penetration. It is to be understood, however, that the less the depth of penetration, the shorter the time between successive applications of light energy necessary to maintain a hair free skin surface. Thus, instead of a month or a week, the time between successive hair removal procedures might be as little as one or two days.

Depth of penetration may also be limited by using lower light intensities. Neutral density or “gray” filters may be used to reduce the intensity of the light applied to the selected skin surfaces.

Listed below are a number of exemplary settings or combinations of operational parameters particularly suitable for professional devices. In these examples, the total times of the pulse sequences are determined by the selected numbers of pulses, the selected pulse durations and the selected inter-pulse intervals. The light intensity may be automatically adjusted by the light generating device if necessary to ensure consistency among the listed parameter settings.

### PROFESSIONAL USE EXAMPLE 1

In a setting or combination of operational parameters suitable for professional use, an incoherent light applicator device for temporary hair removal generates pulses with a pulse number of two, a pulse duration of 7 msec, an inter-pulse interval of 150 msec, a total pulse energy of 60 J/cm², and a spectral distribution of a commercially available flashlamp, including wavelengths between 500 and 1200 nm.

### PROFESSIONAL USE EXAMPLE 2

A slightly higher setting or combination of operational parameters for an incoherent light applicator device involves pulses with a pulse number of two, a pulse duration of 7 msec, an inter-pulse interval of 100 msec, a total pulse energy of 60 J/cm², and a spectral distribution of a commercially available flashlamp, including wavelengths between 500 and 1200 nm.

### PROFESSIONAL USE EXAMPLE 3

A lower setting or combination of operational parameters for an incoherent light applicator device involves pulses with a pulse number of two, a pulse duration of 9 msec, an inter-pulse interval of 100 msec, a total pulse energy of 60 J/cm², and a spectral distribution of a commercially available flashlamp, including wavelengths between 500 and 1200 nm.

### PROFESSIONAL USE EXAMPLE 4

A higher setting or combination of operational parameters for an incoherent light applicator device involves pulses with a pulse number of two, a pulse duration of 9 msec, an inter-pulse interval of 100 msec, a total pulse energy of 100 J/cm², and a spectral distribution of a commercially available flashlamp, including wavelengths between 500 and 1200 nm.

### PROFESSIONAL USE EXAMPLE 5

In a relatively low setting or combination of operational parameters for professional use, an incoherent light applicator device produces pulses with a pulse number of two, a pulse duration of 9 msec, an inter-pulse interval of 200 msec, a total pulse energy of 40 J/cm², and a spectral distribution of a commercially available flashlamp, including wavelengths between 500 and 1200 nm.

### PROFESSIONAL USE EXAMPLE 6

A slightly higher setting or combination of operational parameters for an incoherent light applicator device involves pulses with a pulse number of two, a pulse duration of 5 msec, an inter-pulse interval of 150 msec, a total pulse energy of 40 J/cm², and a spectral distribution of a commercially available flashlamp, including wavelengths between 500 and 1200 nm.

### PROFESSIONAL USE EXAMPLE 7

Another higher setting or combination of operational parameters for an incoherent light applicator device involves pulses with a pulse number of two, a pulse duration of 5 msec, an inter-pulse interval of 150 msec, a total pulse energy of 50 J/cm², and a spectral distribution of a commercially available flashlamp, including wavelengths between 500 and 1200 nm.

An incoherent light applicator device for professional use may also be provided with a low-pass filter, a band-pass filter, or a high-pass filter. A band-pass filter serves to limit the spectral distribution of the generated light pulses to wavelengths in a given band, for instance, between 700 nm and 900 nm. Again, a low-pass filter may be used for transmitting to a skin surface only wavelengths less than a predetermined maximum, such as 900 nm, 750 nm, or 550 nm.

The hair treatment method described above with reference to FIGS. 1 and 2 results not only in a temporary hair removal at an optically treated skin surface, but also retards the growth of hair fibers located at or along that skin surface. By counting the days to hair reappearance after several hair depilation procedures over a course of a few months, it is possible to determine a reduction in hair growth rate owing to the application of electromagnetic radiation. A user who starts using the light application process at one inter-application interval may subsequently use a longer inter-application interval and still maintain a hair-free skin surface. Of course, the degree of hair growth rate reduction will vary from person to person and even from skin location to skin location on the same person. For example, two users initially required to apply the pulsed light energy at intervals of one week in order to prevent the reappearance of hair on the treated hair surface may find that after several months one user need reapply light energy only every two weeks and the other user need reapply light energy only every month.

It is to be noted that a light-pulse treatment method as described herein contemplates multiple passes over any particular skin surface. The selected light treatment parameters may be the same for each pass or may vary from pass to pass. In addition, the passes may follow immediately after one another or may be spaced by an interval during which, for instance, the light treatment device is used to apply light pulses to another area of the user's skin. An advantage of multiple passes is that the rate of power applied to a given skin surface may be reduced relative to that needed for accomplishing the desired hair removal by a single pass or light treatment. Thus, even though the total applied energy may be greater with multiple passes than with a single pass, the energy is spread out over a significantly longer period, thereby posing a reduced risk of damage to the skin. For example, instead of a single pass of 50 Joules/cm², hair could be effectively removed temporarily by two passes of 30 Joules/cm² apiece.

It is to be noted that a light source for generating a preliminary light pulse may be disposed in a location spaced from the primary source 18, 42 of light treatment pulses. The preliminary light source may be disposed in a fixture on a wall or ceiling, or on a separate stand. In any event, the preliminary light source is operatively connected to the control unit 14, 40 so that the timing of the preliminary light bursts or pulses are synchronized to the generation of the treatment pulses. The connection may be a wireless link.

The may be more than one preliminary pulse generator. For example, a plurality of preliminary pulse sources may be disposed in a treatment room. One of those sources may be optionally located on the casing 28, 30 of the light treatment device as discussed above with reference to FIGS. 1 and 2.

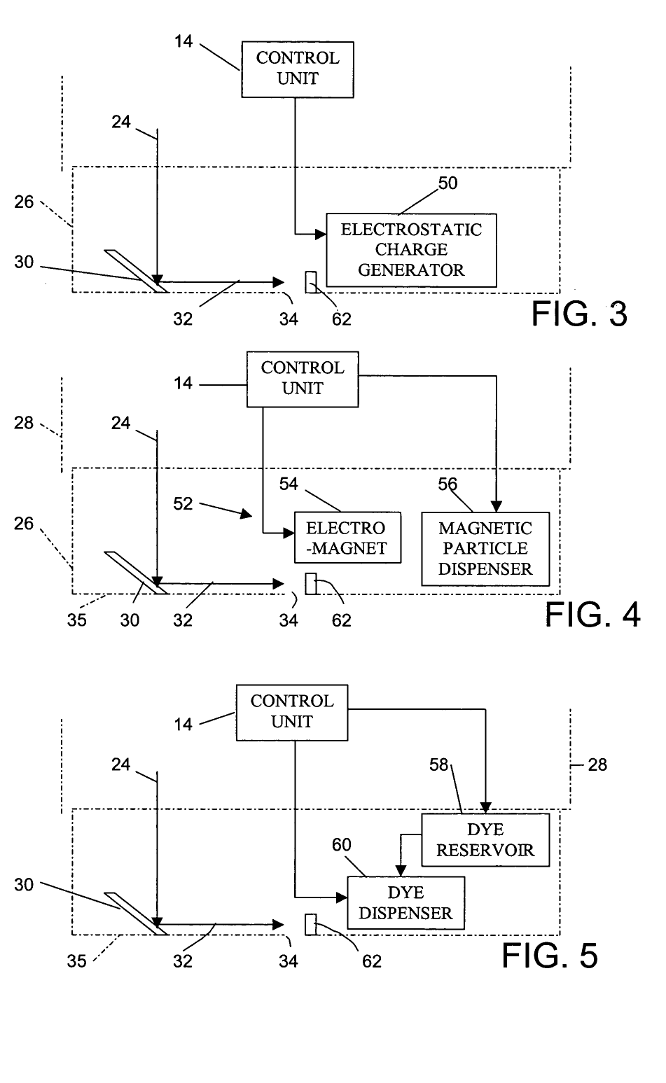

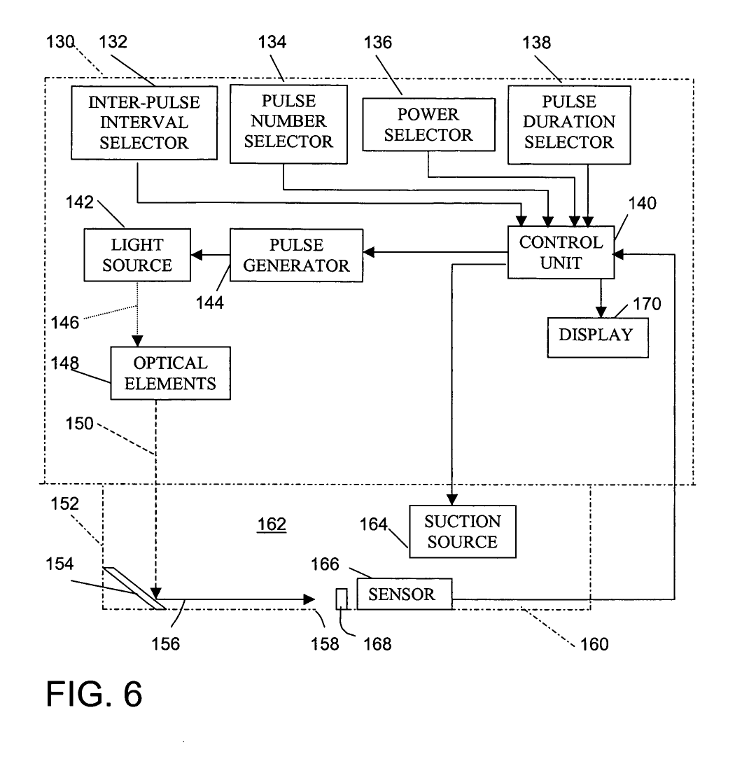

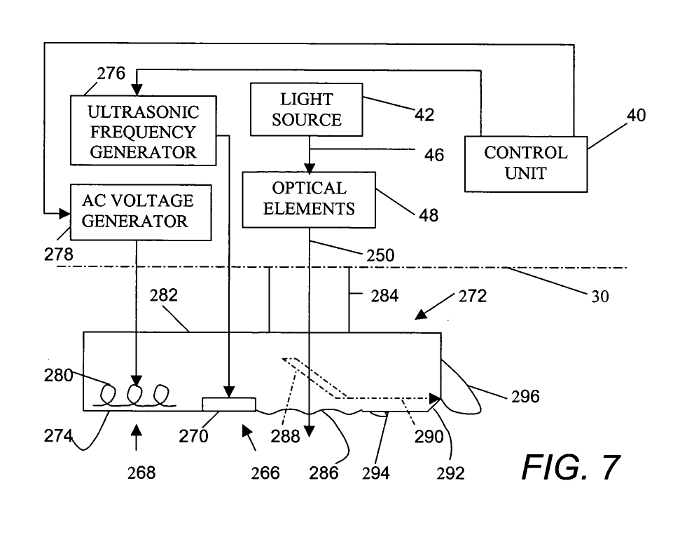

In a specific alternative design, illustrated schematically in FIG. 3, a preliminary light generator 100 with remote operation includes a pulse generator 102 operatively connected at an input to control unit 40 (or 14) via a wireless transmitter 104 and a wireless receiver 106. Transmitter 104 is located, together with control unit 40, in casing 30, while receiver 106 and pulse generator 102 are mounted to a frame 108 of a pair of protective goggles 110. Pulse generator 102 is coupled at an output to one or more light sources 112, 114 exemplarily in the form of light emitting diodes (LEDs). Light sources 112, 114 are arranged about a periphery or edge 116 of a lens 118 made of light-limiting optical material. Depending in part on the shape of lens 118, the light emitted by sources 112, 114 may be confined internally to the lens material by internal reflection. A light-absorbing coating (not shown) may be placed about the periphery or edge 116 of lens 118 for absorbing light that is not internally reflected at the periphery or edge. The intensity of the preliminary light pulses transmitted into lens 118 may be less than in cases where the light source is disposed remotely from the goggles 110, owing in part to the proximity of the sources 112, 114 to the lens 118. The efficacy of the preliminary light pulses is enhanced by the proximity of light sources 112, 114 to the optical material of lens 118.

Each person present in a light treatment room may be provided with a respective pair of goggles 110. The darkening reaction of the light-limiting material of each lens 118 is triggered by light sources 112, 114 disposed on the goggles frame 108 in proximity to the lenses 118.

Although the invention has been described in terms of particular embodiments and applications, one of ordinary skill in the art, in light of this teaching, can generate additional embodiments and modifications without departing from the spirit of or exceeding the scope of the claimed invention.

The present invention is directed primarily to light treatment processes utilizing incoherent light of relatively high intensity. However, there may be skin treatment or other therapeutic applications of light energy where the treatment light is laser light and the present invention may be useful in those laser light processes as well.

Pursuant to the present invention, the preliminary light pulses are typically of incoherent electromagnetic radiation. However, the preliminary light pulses may take the form of laser light. In that case, protective preliminary bursts of laser radiation may be directed along predetermined paths to known locations of target eyewear. Sensors such as cameras and computer implemented recognition software may be used to instantaneously determine and continuously update the known target locations.

There may be applications in which the target optical limiting material is used in a shield or cover other than eyeglasses or goggles. For instance, if an animal is in the light treatment area, the animal may be placed behind a window made of the light limiting material. If a certain skin surface must be available to view during a procedure but should not be exposed to the treatment radiation, that skin surface can be covered by a sheet or guard of the light limited material. For instance, a shield may permit one to visualize a target site and a surrounding area on a baby's face and protect the major skin area while enabling treatment of the target tissues.

Accordingly, it is understood that the drawings and descriptions herein are proffered by way of example to facilitate comprehension of the invention and should not be construed to limit the scope thereof.

1. A light treatment apparatus comprising:
a casing;
a light source disposed in said casing for generating a predetermined number of light pulses of a predetermined duration, light intensity, and total energy;
an applicator element mounted to said casing in optical communication with said light source for directing light from said source to a target;
at least one signal generator for generating and transmitting, to a sensor, at least one trigger signal prior to the generating of said light pulses by said light source; and
a control unit operatively connected to said light source and said signal generator for synchronizing the operation thereof.

2. The apparatus defined in claim 1 wherein said light pulses are primary light pulses of a first predetermined duration, a predetermined first light intensity and said total energy, said signal generator is a preliminary light generator, and said trigger signal is a preliminary light pulse of a predetermined second duration and a second light intensity, said second light intensity being less than said first light intensity and sufficiently low so that said preliminary light pulse poses no substantial risk of damage to retinal receptors.

3. The apparatus defined in claim 2 wherein said preliminary light pulse has a sufficient intensity to activate a light limiting reaction in light-limiting optical material prior to the generating of said primary light pulses.

4. The apparatus defined in claim 3 wherein said light source is a primary light source, said preliminary light generator including a secondary light source different from said primary light source.

5. The apparatus defined in claim 4 wherein said secondary light source includes a light emitting diode.

6. The apparatus defined in claim 3 wherein the light-limiting reaction of said optical material has a predetermined delay or lag time from an initial impingement of light on said sensor to a point where the optical material is sufficient darkened to effectively block light transmission, said preliminary light pulse beginning prior to said primary light pulses by a time at least equal to said delay or lag time.

7. The apparatus defined in claim 6 wherein said preliminary light pulse begins at least one-tenth of a millisecond prior to said primary light pulses.

8. The apparatus defined in claim 3 wherein said light-limiting optical material is in protective eyewear.

9. The apparatus defined in claim 8 wherein said sensor is different from the said light-limiting optical material.

10. The apparatus defined in claim 8 wherein said sensor is said light-limiting optical material.

11. The apparatus defined in claim 2 wherein said light pulses are greater than one in number and have at least one predetermined inter-pulse interval.

12. The apparatus defined in claim 11 wherein said preliminary light pulse is one of a plurality of preliminary light pulses produced by said preliminary light generator, each of said preliminary light pulses beginning prior to a respective one of said primary light pulses.

13. The apparatus defined in claim 12 wherein said preliminary light pulse has a sufficient intensity to activate a light limiting reaction in light-limiting optical material prior to the generating of said primary light pulses, the light-limiting reaction of said optical material having a predetermined delay or lag time from an initial impingement of light on said sensor to a point where the material is sufficient darkened to effectively block light transmission, each of said preliminary light pulses beginning prior to the respective one of said primary light pulses by a time at least equal to said delay or lag time.

14. The apparatus defined in claim 2 wherein said preliminary light pulse is of incoherent light energy.

15. The apparatus defined in claim 1 wherein said trigger signal is transmitted wirelessly to said sensor.

16. The apparatus defined in claim 15, further comprising a wireless transmitter operatively connected to said signal generator.

17. The apparatus defined in claim 15 wherein said trigger signal is a light signal, said sensor being a photodetector.

18. The apparatus defined in claim 1 wherein said light pulses are of incoherent light energy.

19. A light treatment method comprising:
generating a predetermined number of light pulses of a predetermined duration, light intensity, and total energy;
directing said light pulses to a target;
automatically generating at least one trigger signal prior to the generating of said primary light pulses;
transmitting said trigger signal to a sensor prior to the generating of said primary light pulses; and
in response to a receipt of said trigger signal by said sensor, automatically generating a light limiting reaction in a generally transparent light-limiting optical material, thereby preventing transmission of said light pulses through said generally transparent material.

20. The method defined in claim 19 wherein said light pulses are primary light pulses of a predetermined first duration, first light intensity, and said total energy, said trigger signal being at least one preliminary light pulse of a predetermined second duration and second light intensity, said second light intensity being less than said first light intensity and sufficiently low so that said preliminary light pulse poses no substantial risk of damage to retinal receptors.

21. The method defined in claim 20 wherein said preliminary light pulse has a sufficient intensity to trigger said light limiting reaction in said light-limiting optical material prior to the generating of said primary light pulses.

22. The method defined in claim 21 wherein the preliminary light pulse acts directly on said light-limiting optical material to activate a darkening reaction therein.

23. The method defined in claim 21 wherein a sensor different from said light-limiting optical material is responsive to said preliminary light pulse and acts on said light-limiting optical material to activate a darkening reaction therein.

24. The method defined in claim 20 wherein the generating of said primary light pulses includes operating a first light source and the generating of said preliminary light pulse includes operating a second light source different from said first light source.

25. The method defined in claim 20 wherein said preliminary light pulse begins at least one-tenth of a millisecond prior to said primary light pulses.

26. The method defined in claim 20 wherein the directing of said primary light pulses to said target includes directing said light pulses in a first direction, further comprising directing said preliminary light pulse in at least one second direction different from said first direction.

27. The method defined in claim 20 wherein said preliminary light pulse is of incoherent light energy.

28. The method defined in claim 19 wherein said light pulses are of incoherent light energy.

29. The method defined in claim 19 wherein said light-limiting optical material is in protective eyewear.

30. The method defined in claim 19 wherein the light-limiting reaction of said optical material has a predetermined delay or lag time from an initial reception of said trigger signal by said sensor to a point where the material is sufficient darkened to effectively block light transmission, said trigger signal being generated and transmitted prior to said light pulses by a time at least equal to said delay or lag time.

31. The method defined in claim 19 wherein said sensor is said light-limiting optical material.

32. The method defined in claim 19 wherein said sensor is different from said light-limiting optical material, said sensor being operatively connected to said light-limiting optical material for acting on said light-limiting optical material to darken the same.

33. A method for protecting eyes from being damaged by pulsed light, comprising:
providing a window of a generally transparent light-limiting optical material;
disposing said window in front of a set of eyes; and
in response to a high intensity light pulse directed at said window from a side opposite said eyes, automatically generating a light limiting reaction in said generally transparent light-limiting optical material, thereby preventing transmission of said light pulse through said generally transparent material.
